(19) United States
(12) Patent Application Publication
(10) Pub. No.: US 2025/0060257 A1
(43) Pub. Date: Feb. 20, 2025

(54) HEAT MITIGATION FOR MEDICAL DEVICE

(71) Applicant: Welch Allyn, Inc., Skaneateles Falls, NY (US)

(72) Inventors: David M. Fallat, Auburn, NY (US); David M. Babson, Warners, NY (US); Thomas H. Briggs, Frederick, MD (US); Kenneth V. Coon, Jordan, NY (US); Alan S. Knieriem, Baldwinsville, NY (US); Michael T. McMahon, Syracuse, NY (US)

(21) Appl. No.: 18/798,963

(22) Filed: Aug. 9, 2024

Related U.S. Application Data
(60) Provisional application No. 63/519,939, filed on Aug. 16, 2023.

(51) Int. Cl. G01K 3/00; A61B 5/00; A61B 5/01; G01K 1/024; G01K 13/20; G16H 40/40

(52) U.S. Cl. CPC G01K 3/005; A61B 5/01; A61B 5/742; G01K 1/024; G01K 13/20; G16H 40/40; A61B 2560/0214

(57) ABSTRACT

A medical device for capturing one or more physiological variables of a patient is described. The medical device includes a housing, a display device mounted on the housing, and ports on the housing for connecting one or more peripheral sensors. The medical device receives data from the peripheral sensors, processes the data to calculate at least one physiological variable, and displays the at least one physiological variable on the display device. The medical device monitors an internal temperature inside the housing, determines whether the internal temperature exceeds a predefined threshold, and when the internal temperature exceeds the predefined threshold, perform one or more actions to lower the internal temperature.

### BACKGROUND

Modern electronic medical devices typically include powerful processors that drive large displays, and can also include rechargeable batteries. Such internal components typically generate heat which can affect the performance of the medical devices. Typically, the housings of such medical devices include a fan to cool down these internal components. However, fans can generate noise. Additionally, fans can cause particles such as dirt and dust to get into the housings of such medical devices, which can plug intake and exhaust vents. This can result in increased internal temperatures inside the housings of such medical devices. In some instances, the increased internal temperatures can eventually make such medical devices inoperable.

### SUMMARY

In general terms, the present disclosure relates to heat mitigation for a medical device. In one possible configuration, the medical device provides a technical effect by performing one or more actions to lower an internal temperature when the internal temperature is determined to exceed a predefined threshold. Various aspects are described in this disclosure, which include, but are not limited to, the following aspects.

One aspect relates to a medical device for capturing one or more physiological variables of a patient, the medical device comprising: a housing; a display device mounted on the housing; ports on the housing for connecting one or more peripheral sensors; at least one processing device; and at least one computer readable data storage device storing software instructions that, when executed by the at least one processing device, cause the at least one processing device to: receive data from the peripheral sensors; process the data to calculate at least one physiological variable; display the at least one physiological variable on the display device; monitor an internal temperature inside the housing; determine whether the internal temperature exceeds a predefined threshold; and when the internal temperature exceeds the predefined threshold, perform one or more actions to lower the internal temperature.

Another aspect relates to a method of capturing one or more physiological variables of a patient, the method comprising: receiving data from a peripheral sensor; processing the data to calculate a physiological variable by using a computing device mounted inside a housing; displaying the physiological variable on a display device; monitoring an internal temperature inside the housing; determining whether the internal temperature exceeds a predefined threshold; performing one or more actions to lower the internal temperature when the internal temperature exceeds the predefined threshold; and generating an alarm identifying the one or more actions performed to lower the internal temperature.

Another aspect relates to a non-transitory computer readable storage medium storing instructions, which when executed by a computing device, cause the computing device to: receive data from a peripheral sensor; process the data to calculate at least one physiological variable by using a computing device mounted inside a housing; display the at least one physiological variable on a display device; monitor an internal temperature inside the housing; determine whether the internal temperature exceeds a predefined threshold; and when the internal temperature exceeds the predefined threshold, perform one or more actions to lower the internal temperature.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 is a front isometric view of a medical device that can be used to monitor one or more physiological variables of a subject.

FIG. 2 is a rear isometric view of the medical device of FIG. 1.

FIG. 3 is a front view of the medical device of FIG. 1.

FIG. 4 is a rear view of the medical device of FIG. 1.

FIG. 5 is a top view of the medical device of FIG. 1.

FIG. 6 is a bottom view of the medical device of FIG. 1.

FIG. 7 is a left side view of the medical device of FIG. 1.

FIG. 8 is a right side view of the medical device of FIG. 1.

FIG. 9 is a cross-sectional top view of the medical device of FIG. 1.

FIG. 10 is a cross-sectional right side view of the medical device of FIG. 1.

FIG. 11 shows an example of a main circuit board of the medical device of FIG. 1.

FIG. 12 is a map of temperature distributions on the main circuit board of FIG. 11.

FIG. 13 is another map of temperature distributions inside a housing of the medical device of FIG. 1.

FIG. 14 is an isometric view of a heat sink on the main circuit board of FIG. 11.

FIG. 15 is a front isometric view of the heat sink of FIG. 14.

FIG. 16 is a rear isometric view of the heat sink of FIG. 14.

FIG. 17 schematically illustrates an example of a method of mitigating internal heat generated by the medical device of FIG. 1.

FIG. 18 schematically illustrates examples of actions that can be performed to mitigate the heat generated by the medical device of FIG. 1.

FIG. 19 illustrates an example of a user interface displayed on a display device of the medical device of FIG. 1.

FIG. 20 schematically illustrates another example of a method of mitigating internal heat generated by the medical device of FIG. 1.

FIG. 21 schematically illustrates an exemplary architecture of a computing device of the medical device of FIG. 1.

FIG. 22 schematically illustrates an example of a charge circuit for a rechargeable battery of the medical device of FIG. 1.

### DETAILED DESCRIPTION

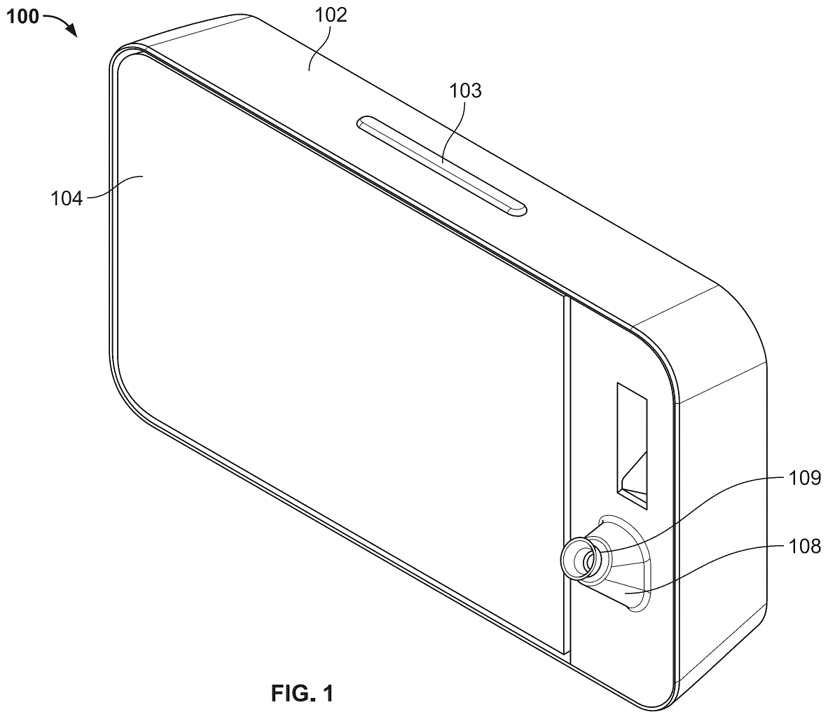

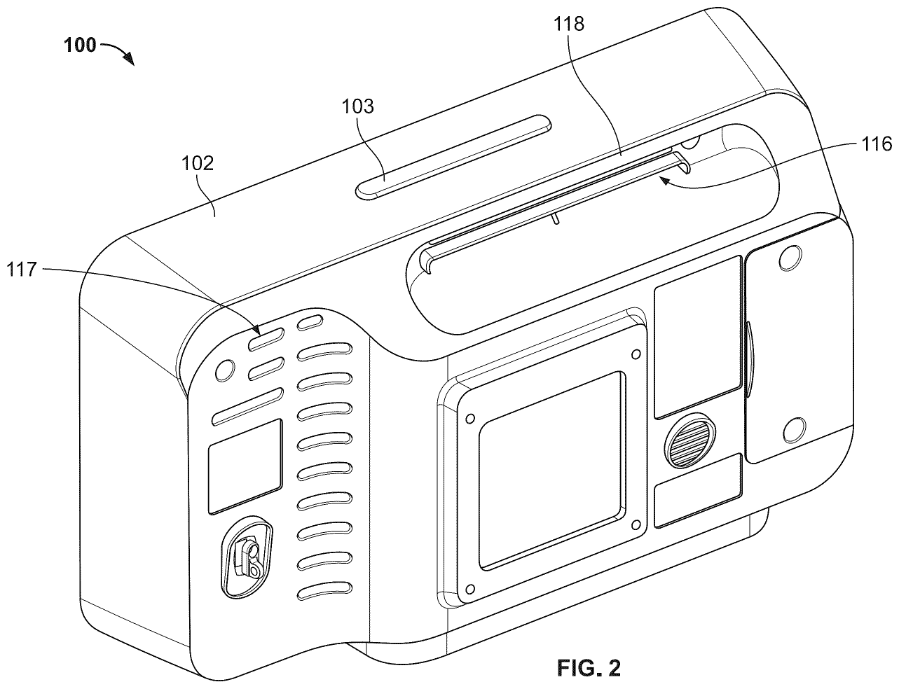

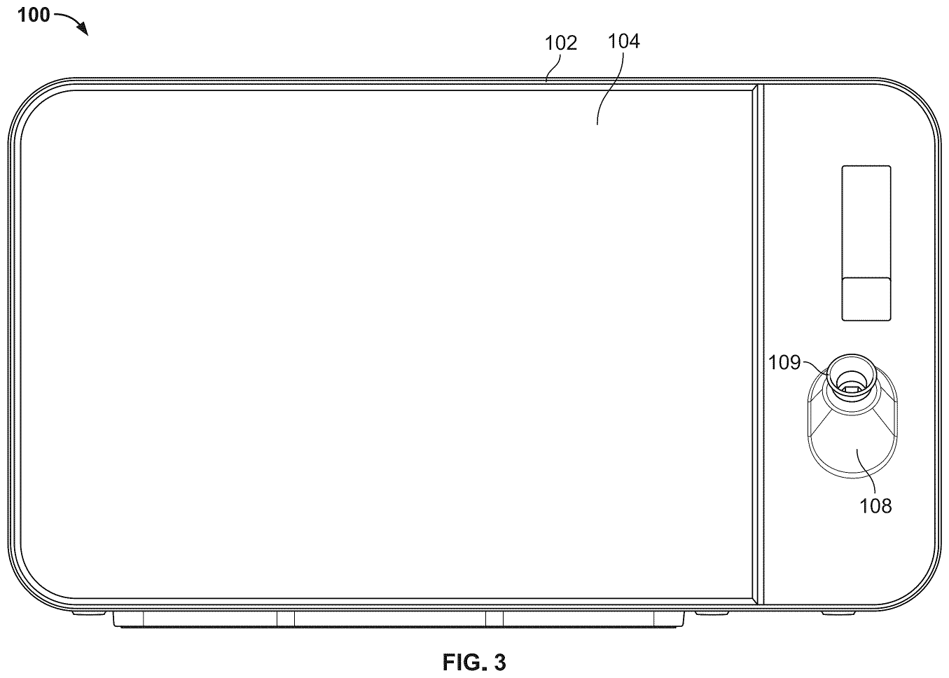

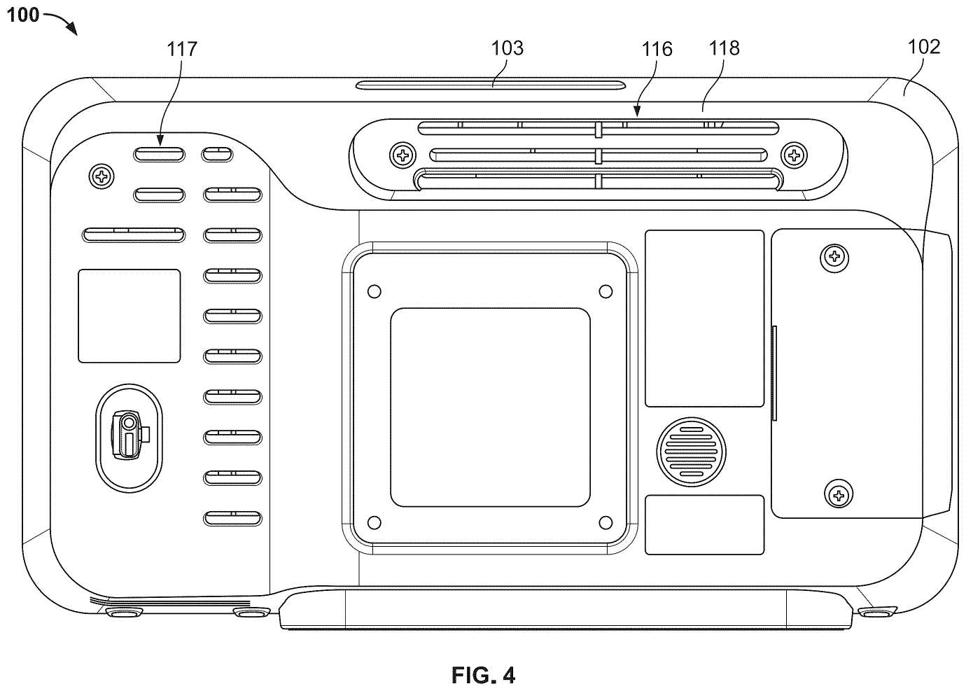

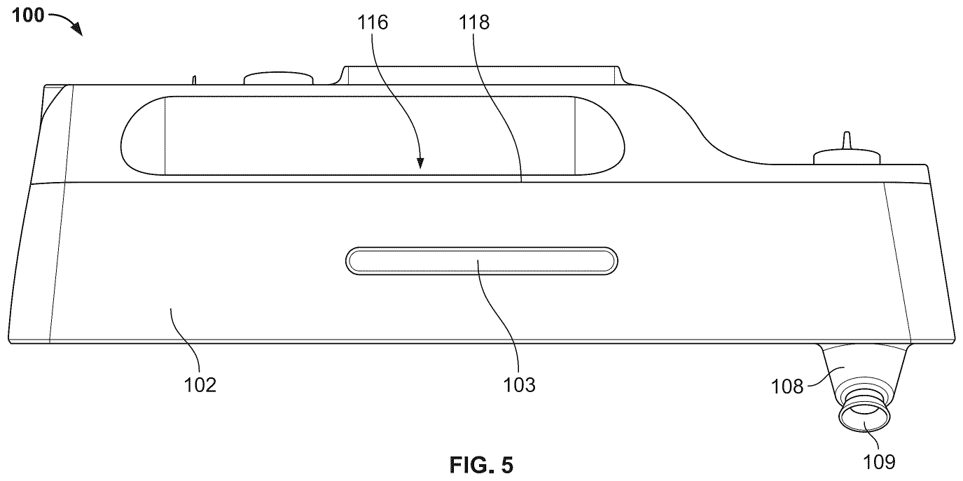

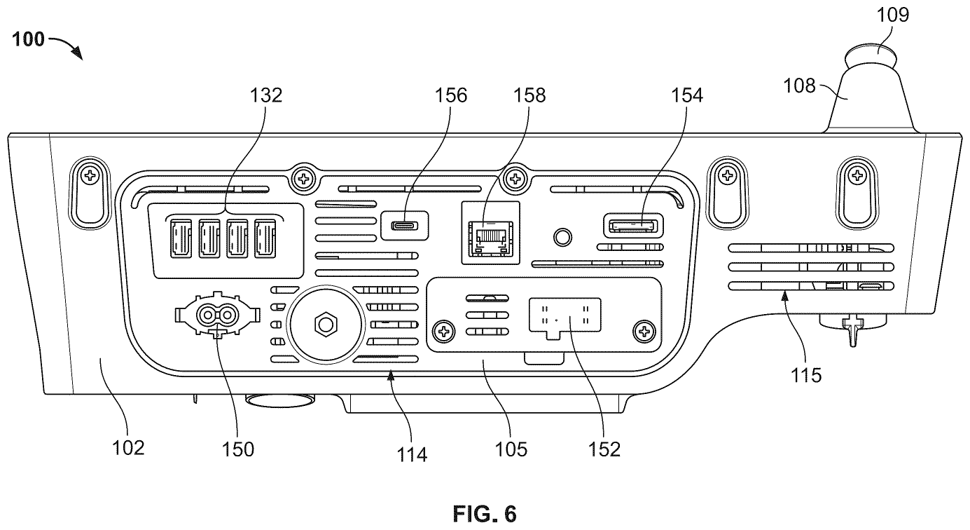

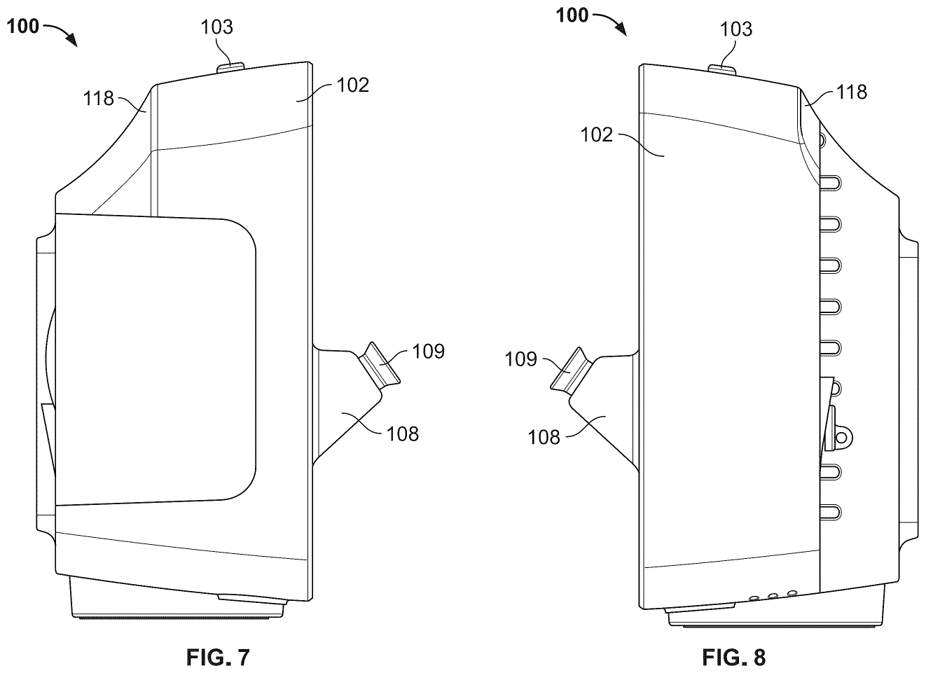

FIG. 1 is a front isometric view of a medical device 100 that can be used to monitor one or more physiological variables of a subject. FIG. 2 is a rear isometric view of the medical device 100. FIGS. 3 and 4 are respective front and rear views of the medical device 100. FIGS. 5 and 6 are respective top and bottom views of the medical device 100. FIGS. 7 and 8 are respective left side and right side views of the medical device 100.

The medical device 100 is designed for use in a clinical setting. In some examples, the medical device 100 is a spot monitor that can be used for episodic monitoring of one or more physiological variables of a patient admitted to a medical facility such as a hospital, medical clinic, and the like. In some further examples, the medical device 100 can also be used for continuous monitoring of one or more physiological variables.

As will be described in more detail, the medical device 100 controls an internal temperature inside a housing 102 of the device by providing heat mitigation through mechanical, electrical, and software means. In particular, the medical device 100 controls the internal temperature inside the housing 102 by providing heat mitigation without using a fan.

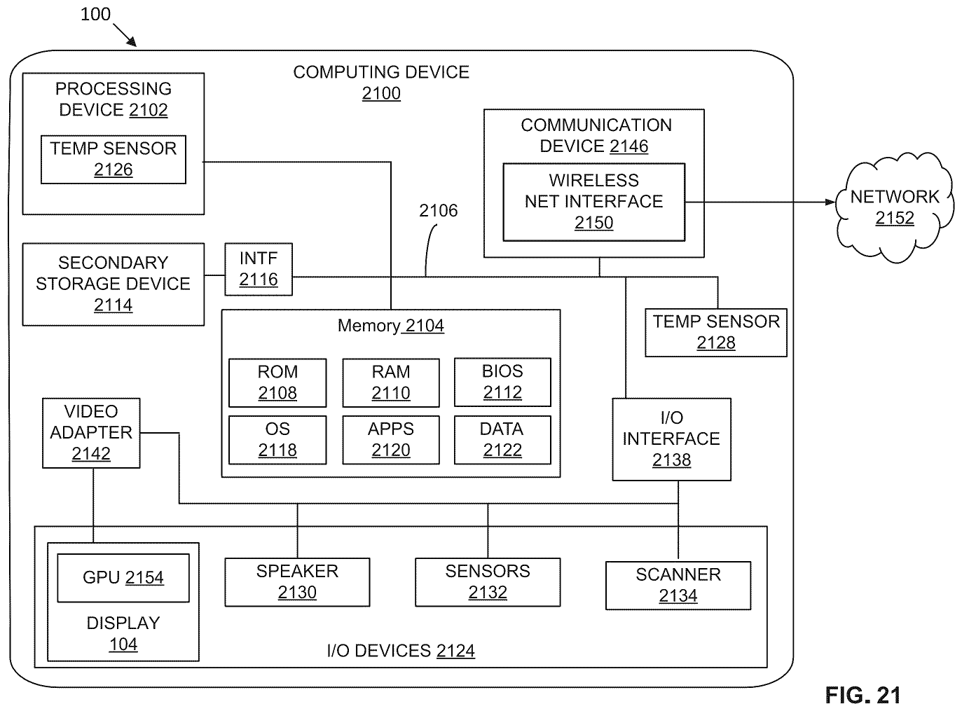

The medical device 100 includes one or more peripheral sensors 2132 (see FIG. 21) that can be used to measure the one or more physiological variables including, without limitation, blood pressure, blood oxygen saturation (SpO2), pulse rate, body temperature, and respiration rate. The peripheral sensors 2132 can include a thermometer module 108 to measure body temperature, a non-invasive blood pressure cuff to measure systolic and diastolic blood pressure, a pulse oximeter to measure blood oxygen saturation and pulse rate, and can further include additional types of sensors for measuring additional types of physiological variables.

The peripheral sensors 2132 can included cables that terminate into connectors that can be plugged into various ports included on the housing 102 for transmission of data via a wired connection to a computing device 2100 (see FIG. 21) of the medical device 100. For example, as shown in FIG. 6, the housing 102 can include a first port 150 for connecting the non-invasive blood pressure cuff, a second port 152 for connecting the pulse oximeter, a third port 154 for connecting the medical device 100 to a printer, one or more universal serial bus (USB) ports 132, a USB-C port 156, and a registered jack (RJ) interface 158. In some instances, the peripheral sensors 2132 can wirelessly transmit the data to the computing device 2100.

The thermometer module 108 is integrated with the medical device 100. The thermometer module 108 includes a port 109 for housing a handheld probe that can be orally inserted to take a temperature reading of a patient. Alternatively, the handheld probe that can be configured for insertion into an ear of the patient to take a temperature reading.

The computing device 2100 receives the data from the peripheral sensors 2132 for processing. For example, the computing device 2100 can calculate numerical values and/or waveforms for the one or more physiological variables based on the data received from the peripheral sensors 2132. The computing device 2100 can display the numerical values and/or waveforms of the one or more physiological variables on a display device 104.

The medical device 100 can also include a scanner 2134 (see FIG. 21) that can be used to scan machine-readable labels. For example, the scanner 2134 can be used to scan machine-readable labels that are printed on wristbands or other articles worn by patients to identify the patients. The scanner 2134 can also be used to scan machine-readable labels on articles such as badges worn by caregivers, on medications administered to the patient, and on other medical devices and equipment. The scanner 2134 can include a cable that terminates into a connector that can be plugged into a port included on the housing 102 of the medical device 100. Alternatively, the scanner can wirelessly communicate with the medical device 100.

The medical device 100 can also include a radio-frequency identification (RFID) reader that can be used to detect RFID tags attached to objects in close proximity to the medical device 100. The medical device 100 can be configured for wireless communications via Wi-Fi, Bluetooth, RFID, near-field communications (NFC), and other wireless technologies.

As shown in FIGS. 1-8, the medical device 100 includes the display device 104, which is mounted to a front portion of the housing 102. The display device 104 is a touch sensitive touchscreen that receives inputs from a user of the medical device 100 such as a caregiver including physicians, clinicians, nurses, and other trained medical professionals.

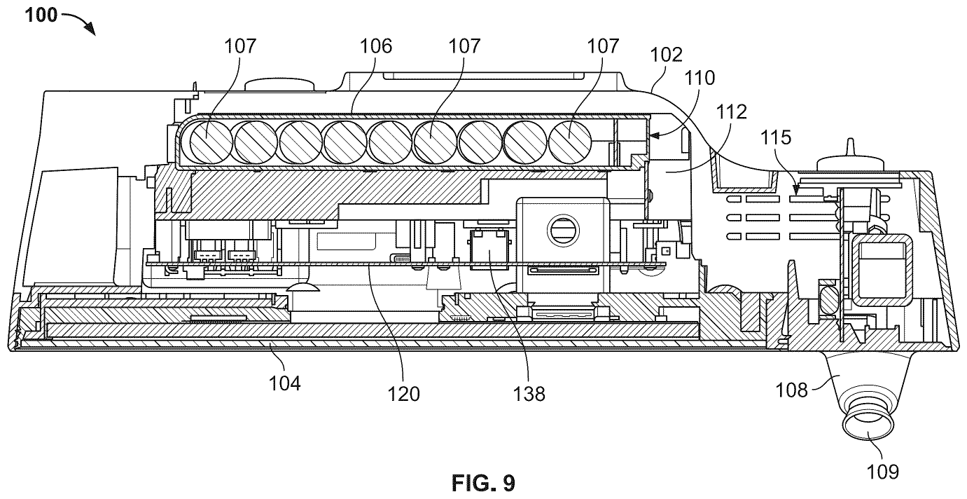

FIG. 9 is a cross-sectional top view of the medical device 100. As shown in FIG. 9, a rechargeable battery 106 is housed inside the housing 102 of the medical device 100. The rechargeable battery 106 can be used to power the electrical components of the medical device 100 (e.g., the display device 104, the computing device 2100, the peripheral sensors 2132 including the thermometer module 108, and the like) when the medical device 100 is not plugged into a mains electricity power supply such as a wall socket. The rechargeable battery 106 is designed to provide enough power to last an entire shift of a user of the medical device 100 such as a caregiver (e.g., a nurse) on shift in a medical facility (e.g., a hospital). In some examples, the rechargeable battery 106 is designed to provide up to eight hours of power supply to the electrical components of the medical device 100. The rechargeable battery 106 includes a plurality of cells 107. In the example shown in FIG. 9, the rechargeable battery includes nine cells. The plurality of cells 107 can include lithium-ion cells, or similar types of battery cells.

The rechargeable battery 106 generates heat, especially when being recharged, such as when the medical device 100 is plugged into a mains electricity power supply. Also, the rechargeable battery 106 can generate heat when supplying electrical power to the electrical components of the medical device 100 including electrical components mounted on a main circuit board 120, and the peripheral sensors 2132 including the thermometer module 108.

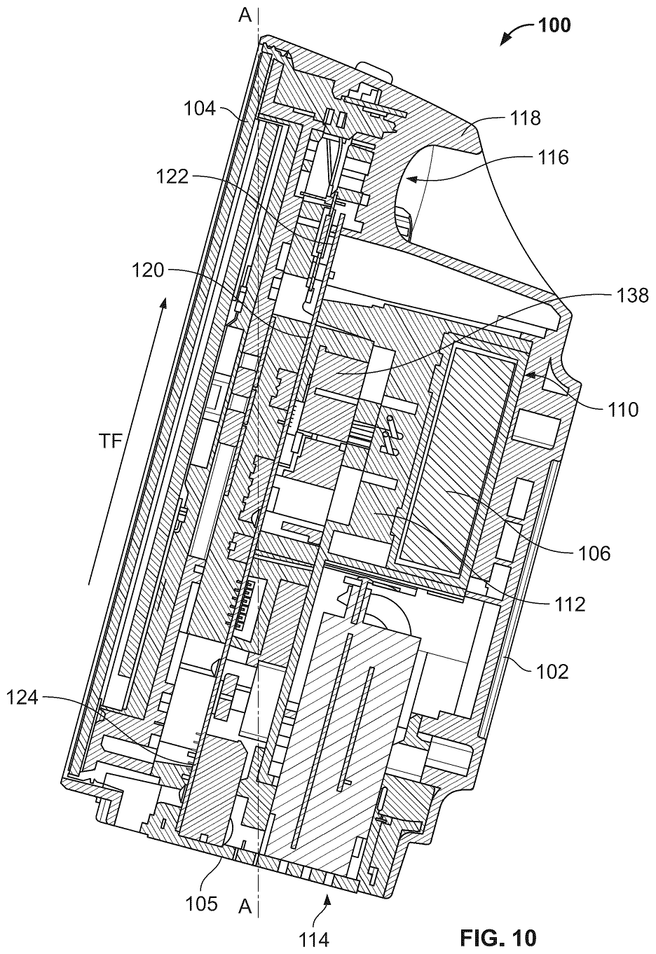

FIG. 10 is a cross-sectional right side view of the medical device 100. As shown in FIGS. 9 and 10, the rechargeable battery 106 is housed inside a compartment 110 defined between the housing 102 and an interior divider 112. The interior divider 112 physically separates the rechargeable battery 106 from the main circuit board 120 of the medical device 100. This can prevent heat generated by the rechargeable battery 106 from reaching the main circuit board 120, which is mounted inside the housing 102. This can help maintain a lower temperature around the main circuit board 120, and thereby improve the performance of the medical device 100 such as by preventing malfunctioning that can occur due to excess heat interfering with the performance of the main circuit board 120 inside the housing 102.

As described above, the medical device 100 provides heat mitigation through mechanical means. The mechanical means include optimizing the design of the housing 102 to improve a thermal flow TF of a conduit such as air for cooling internal components housed inside the medical device 100 such as the main circuit board 120. For example, the housing 102 includes intake vents 114 (see FIGS. 6 and 10) that are positioned across a bottom surface 105 of the housing 102. The housing 102 further includes exhaust vents 116 (see FIGS. 2, 4, and 10) that are positioned toward a top portion of the housing 102. The intake vents 114 have a shape, a size, and a placement/location that are optimized for increasing air flow into the housing 102, and the exhaust vents 116 have a shape, a size, and a placement/location that are optimized for increasing air flow out of the housing 102.

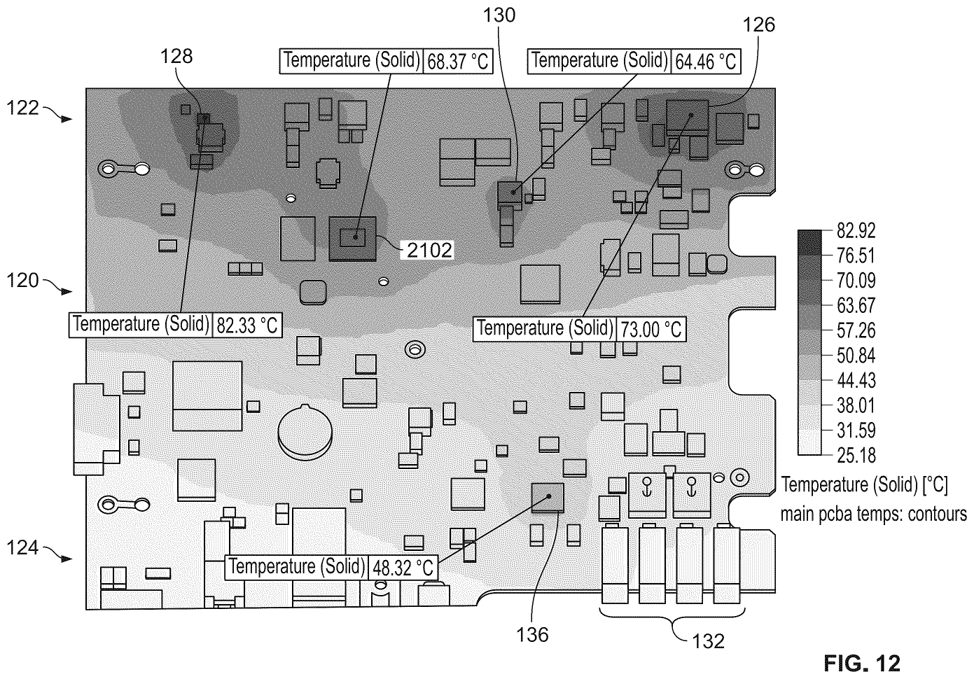

As shown in FIG. 10, the main circuit board 120 is positioned parallel to the thermal flow TF such that cool air enters the housing 102 through the intake vents 114, the air passes around the main circuit board 120 causing convection of heat away from the main circuit board 120 with minimal obstructions, and causing the heat to exit the housing 102 through the exhaust vents 116. The thermal flow TF of the air through the housing 102 is shown in more detail in FIG. 12, which is described in more detail further below. Advantageously, the thermal flow TF of the air that results from the arrangement of the main circuit board 120 mounted inside the housing 102 relative to the intake vents 114 positioned at the bottom of the housing 102, and the exhaust vents 116 positioned toward the top portion of the housing 102, can eliminate the need for a fan for cooling the internal components housed inside the housing 102.

In the example illustrated in FIGS. 1-10, the exhaust vents 116 are set back and positioned under a handle 118 on the housing 102 of the medical device 100. In this example, the handle 118 provides two separate functions. The handle 118 provides a primary function of allowing a user of the medical device 100 to grip the housing 102 such as to physically move the medical device 100 from one location to another, or to adjust an angle of the medical device 100 relative to a support surface or structure such as for tilting the display device 104.

Additionally, the handle 118 provides a secondary function of providing a canopy over the exhaust vents 116 to prevent liquid such as water from entering inside the housing 102, which can cause damage to the internal components housed inside the medical device 100 such as the main circuit board 120. The handle 118 does not interfere with or obstruct the thermal flow TF of air exiting the exhaust vents 116 for cooling the components inside the housing 102.

As shown in FIGS. 2, 4, and 9, the housing 102 includes secondary intake vents 115 and secondary exhaust vents 117 that mitigate heat generated by the thermometer module 108. Cool air enters the housing 102 through the secondary intake vents 115, which are positioned on a bottom portion of the housing 102, the air passes over internal components of the thermometer module 108 allowing efficient convection across the components with minimal obstructions, and hot air then exits the housing 102 through the secondary exhaust vents 117, which are positioned toward the top portion of the housing 102. The secondary intake vents 115 and the secondary exhaust vents 117 provide dedicated heat mitigation for the thermometer module 108 of the medical device 100 which, as described above, is integrated with the medical device 100.

As described above, the medical device 100 provides further heat mitigation through electrical means. As will now be described in more detail, the electrical means for providing heat mitigation inside the medical device 100 can include optimizing the layout of components on the main circuit board 120. The layout of the main circuit board 120 is optimized by positioning components that generate heat toward a top portion of the main circuit board 120, while positioning components that do not generate heat toward a bottom portion of the board.

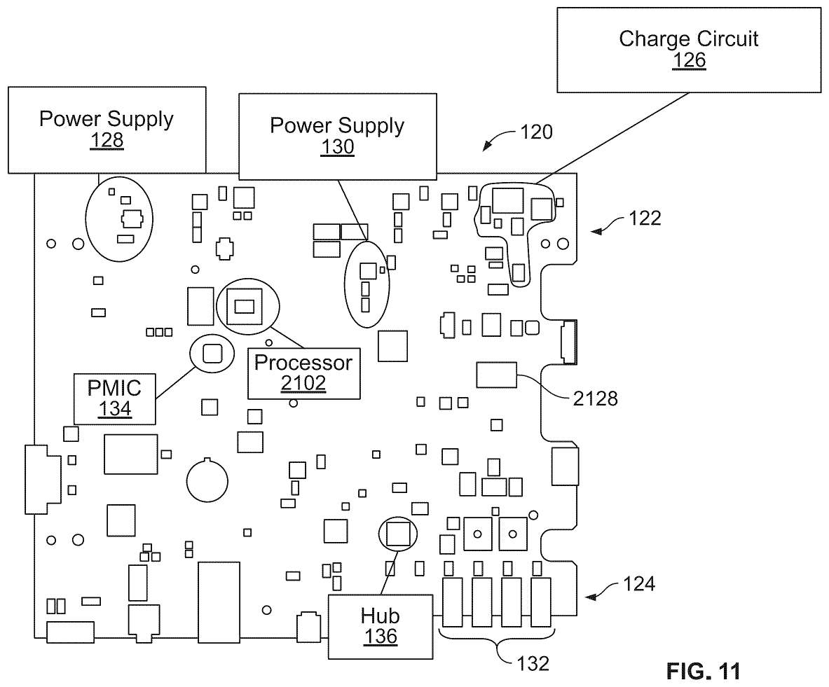

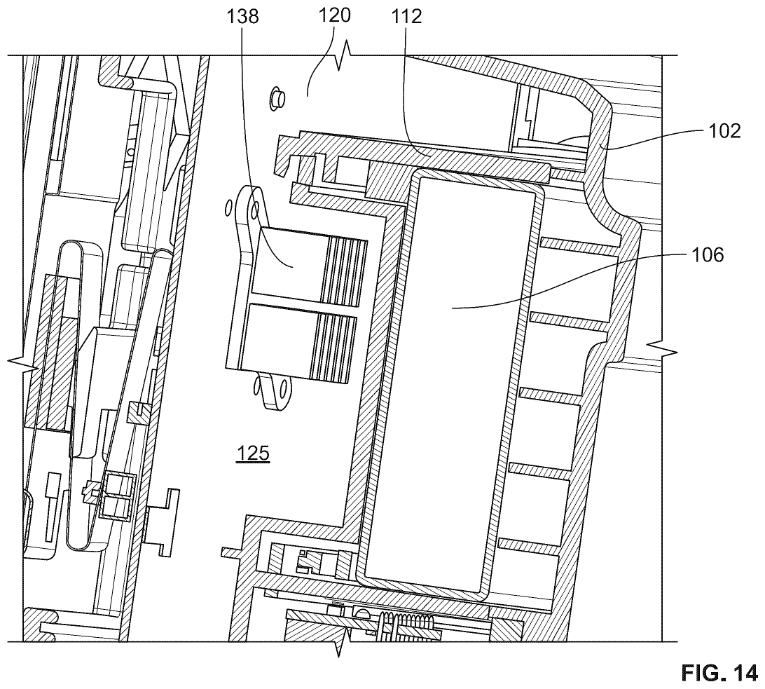

FIG. 11 shows an example of the main circuit board 120 of the medical device 100. Referring now to FIGS. 10 and 11, the main circuit board 120 is positioned vertically inside the housing 102. In the example of FIG. 10, the main circuit board 120 is positioned substantially parallel with the display device 104. The main circuit board 120 includes a top portion 122 which is arranged near the exhaust vents 116, and a bottom portion 124 which is arranged near the intake vents 114 when the main circuit board 120 is mounted inside the housing 102 of the medical device 100. The main circuit board 120 includes a front surface 123 facing the display device 104, and a rear surface 125 (see FIG. 14) facing in an opposite direction.

As described above, the handle 118 on the housing 102 allows a user of the medical device 100 to grip the housing 102 such as to adjust an angle of the housing 102 relative to a support surface or structure such as for tilting the display device 104. When the housing 102 is tilted, the main circuit board 120 (which is positioned substantially parallel with the display device 104) is also tilted with respect to an axis AA (see FIG. 10) orthogonal to the support surface or structure that supports the housing 102. In this arrangement, a surface area of the main circuit board 120 is increased relative to the thermal flow TF of air through the inside of the housing 102 from the intake vents 114 to the exhaust vents 116. This can further increase the cooling of the main circuit board 120, and further mitigate heat generation.

Referring now to FIG. 11, the main circuit board 120 includes a layout where components that generate a large amount of heat are positioned toward the top portion 122 of the main circuit board 120, and components that generate a minimal amount of heat or no heat are positioned toward the bottom portion 124 of the main circuit board 120. For example, the components are mounted on the main circuit board 120 in an order defined by components generating the least amount of heat positioned at the bottom portion 124, and components generating the largest amount of heat positioned toward the top portion 122.

In the example provided in FIG. 11, the components that generate the largest amount of heat such as a charge circuit 126 for the rechargeable battery 106, a power supply 128 for the display device 104, a processing device 2102, a power supply 130 for universal serial bus (USB) ports 132, and a power management integrated circuit (PMIC) 134 for the processing device 2102 are positioned toward the top portion 122 of the main circuit board 120. The components that generate a minimal amount of heat or no heat such as the USB ports 132 and a hub 136 for the USB ports 132 are positioned toward the bottom portion 124 of the main circuit board 120.

As an illustrative example, the charge circuit 126 generates the largest amount of heat (about 2.22 W), followed by the power supply 128 (about 1.8 W), followed by the processing device 2102 (about 1.4 W), and followed by the power supply 130 (about 1 W). The USB ports 132 produce minimal to no heat, while the hub 136 produces minimal heat (about 0.89 W).

FIG. 12 is a map of temperature distributions on the main circuit board 120 during use of the medical device 100. As shown in FIG. 12, on the top portion 122 of the main circuit board 120, the heat generated by the charge circuit 126 causes a localized temperature of about 73° C. (about 163° F.), the heat generated by the power supply 128 causes a localized temperature of about 82° C. (about 180° F.), the heat generated by the processing device 2102 causes a localized temperature of about 68° C. (about 154° F.), and the heat generated by the power supply 130 causes a localized temperature of about 64° C. (about 147° F.). As further shown in FIG. 12, on the bottom portion 124 of the main circuit board 120, the heat generated by the hub 136 causes a localized temperature of about 48° C. (about 118° F.), and the heat generated by the USB ports 132 causes a localized temperature of about 25° C. (about 77° F.), such that the temperature of the bottom portion 124 of the main circuit board 120 is significantly less than the top portion 122.

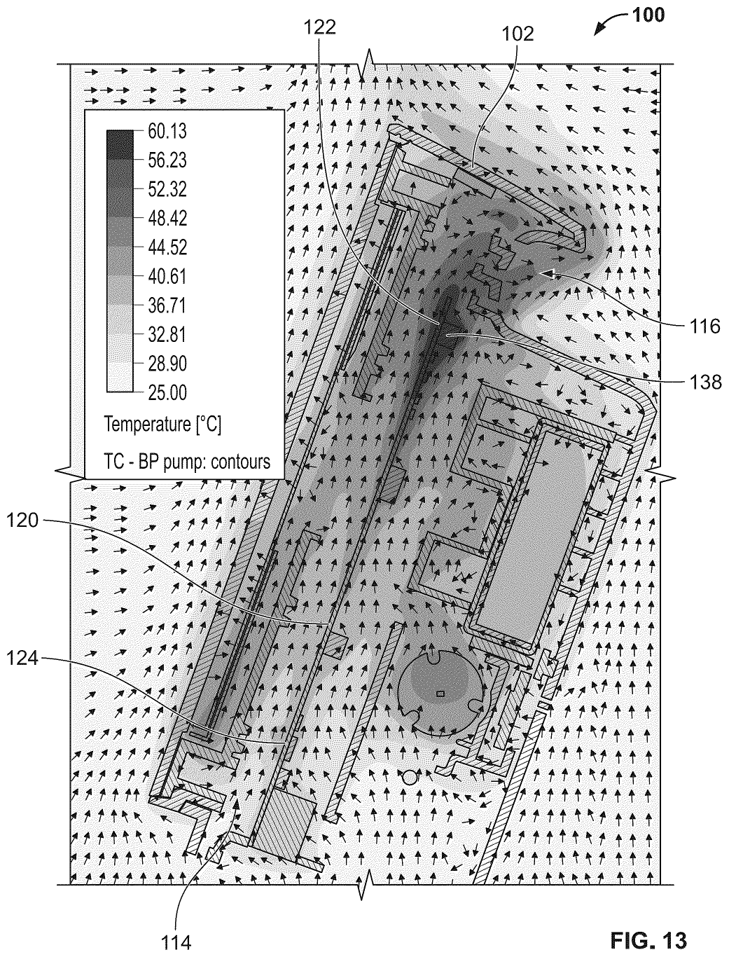

FIG. 13 is another map of temperature distributions inside the housing 102 of the medical device 100. As shown in FIG. 13, cool ambient air enters the housing 102 via the intake vents 114 on the bottom of the medical device 100, and travels through the housing 102 allowing efficient convection across the main circuit board 120 with minimal obstructions, allowing the hot air to exit the housing 102 through the exhaust vents 116 at the top of the medical device 100. Also, the components on the main circuit board 120 having the highest temperature are positioned toward the top portion 122 of the main circuit board 120, such that they are positioned proximate the exhaust vents 116. This provides an improved thermal flow for transferring the heat generated from these components outside of the housing 102, and thereby providing heat mitigation for the internal components of the medical device 100 without using a fan.

FIG. 14 is an isometric view of a heat sink 138 on the main circuit board 120, which is shown mounted inside the housing 102 of the medical device 100. The heat sink 138 is an additional electrical means of providing heat mitigation. The heat sink 138 is mounted to the top portion 122 of the main circuit board 120 to transfer thermal energy from the top portion 122 of the main circuit board 120 to the cool ambient air that enters the housing 102 from the bottom and flows to the top before exiting through the exhaust vents 116. In some examples, the heat sink 138 mounts over the processing device 2102 on the main circuit board 120.

As further shown in examples of FIGS. 11, 13, and 14, the heat generating components are mounted on the front surface 123 of the main circuit board 120, while the heat sink 138 is mounted on the rear surface 125 of the main circuit board 120. Also, the interior divider 112 physically separates the heat sink 138 from the rechargeable battery 106 such that the flow of air over the heat sink 138 is not obstructed by the rechargeable battery 106.

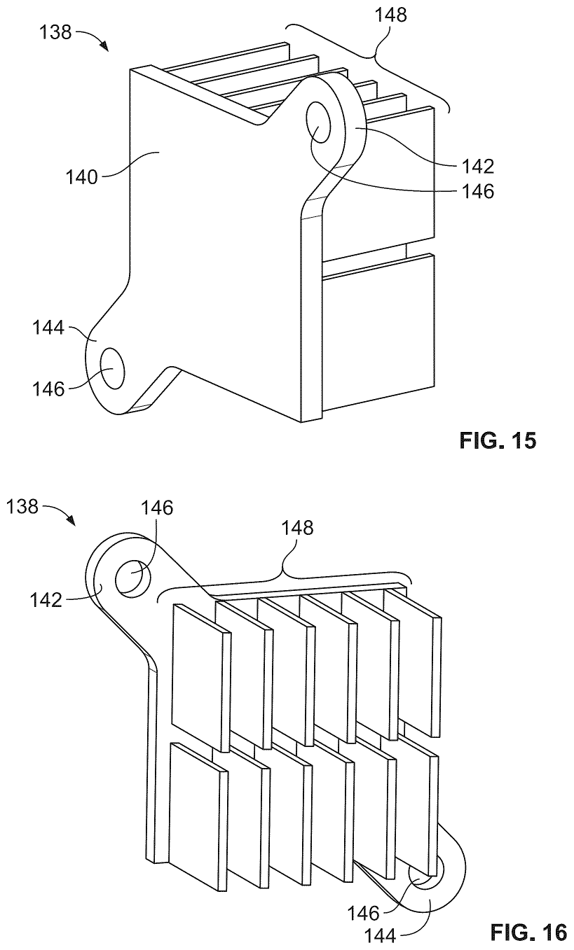

FIGS. 15 and 16 are respective front and rear isometric views of the heat sink 138. As shown in FIGS. 15 and 16, the heat sink 138 includes a body 140 having first and second arms 142, 144 that each include an aperture 146 for receiving a fastener such as a screw to mechanically attach the heat sink 138 to the main circuit board 120. The body 140 further includes a plurality of fins 148 that increase the external surface area of the heat sink 138 to improve the transfer of thermal energy from the main circuit board 120 to the thermal flow TF of air through the interior of the housing 102 of the medical device 100 (see also FIG. 13).

As described above, the medical device 100 provides further heat mitigation through software means. As will now be described in more detail, the software means can include controlling one or more of the components that generate heat on the main circuit board 120 to provide heat mitigation inside the housing 102 of the medical device 100.

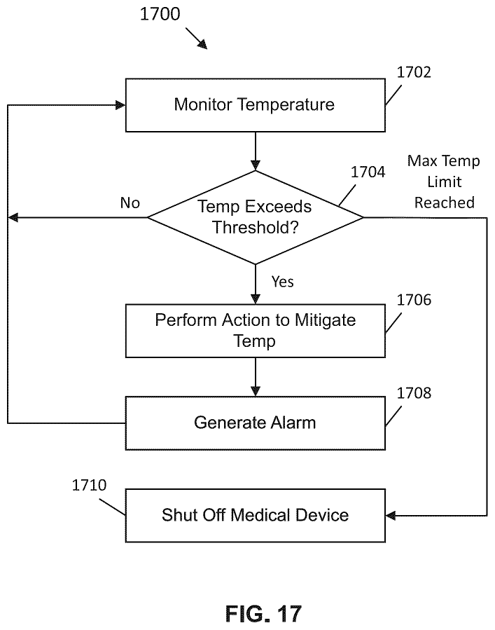

FIG. 17 schematically illustrates an example of a method 1700 of mitigating internal heat generated by the medical device 100. The method 1700 includes an operation 1702 of monitoring an internal temperature of the medical device 100. The internal temperature can be monitored using an internal temperature sensor 2126 of the processing device 2102 (see FIG. 21), and/or a temperature sensor 2128 mounted on a main circuit board 120 (see FIG. 11).

The method 1700 includes an operation 1704 of determining whether the internal temperature exceeds one or more predefined thresholds. When it is determined that the internal temperature does not exceed the one or more predefined thresholds (i.e., “No” in operation 1704), the method 1700 returns to operation 1702 of monitoring an internal temperature of the medical device 100. When it is determined that the internal temperature does exceed the one or more predefined thresholds (i.e., “Yes” in operation 1704), the method 1700 proceeds to an operation 1706 of performing one or more actions to lower the internal temperature.

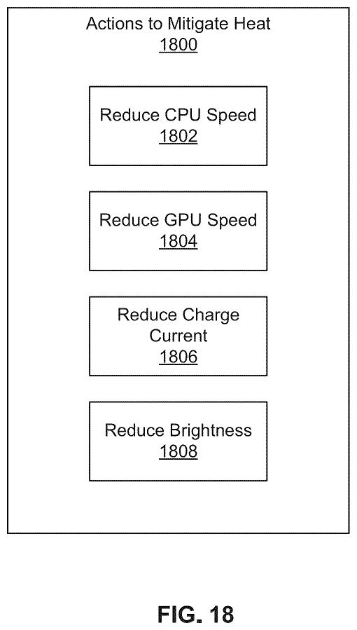

FIG. 18 schematically illustrates examples of actions 1800 that can be performed in operation 1706 to lower the internal temperature of the medical device 100. The actions 1800 can include a first action 1802 of reducing the clock speed (also “clock rate” or “frequency”) of the processing device 2102; a second action 1804 of reducing the clock speed of a graphics processing unit (GPU) 2154 of the display device 104 (see FIG. 21); a third action 1806 of reducing a charge current supplied by the charge circuit 126 for recharging the rechargeable battery 106; and a fourth action 1808 of reducing the brightness of the display device 104.

Referring back to FIG. 17, the method 1700 can further include an operation 1708 of generating an alarm to notify a user of the medical device of the one or more actions performed in operation 1706. The alarm generated in operation 1708 can include a visual alarm such as an alert or notification on the display device 104 to notify a user of the medical device 100 that the temperature inside the medical device 100 exceeds a threshold. The visual alarm displayed on the display device 104 may also identify the one or more actions performed in operation 1706.

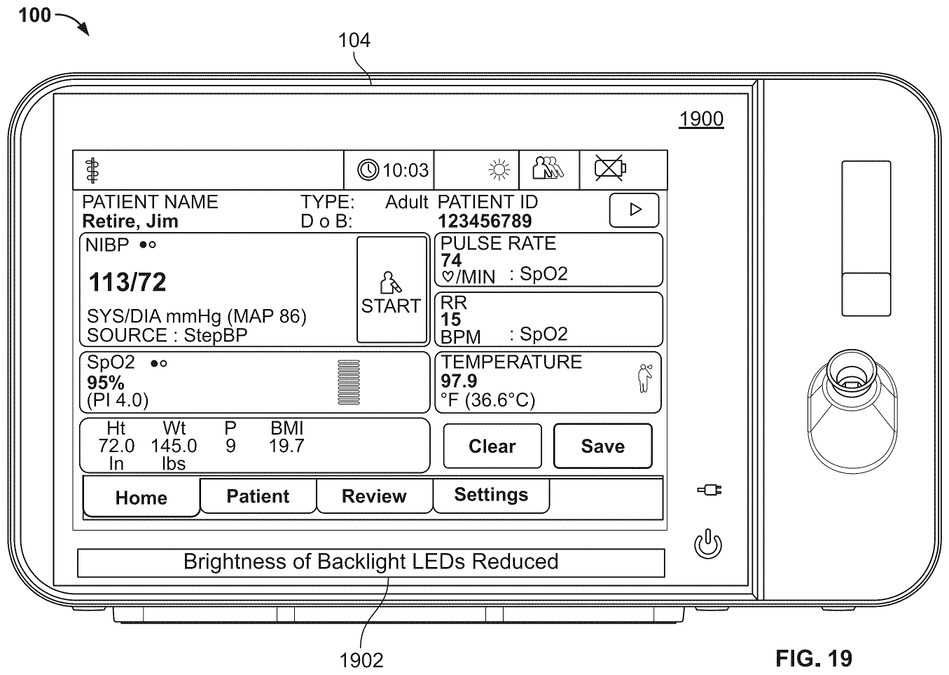

FIG. 19 illustrates an example of a user interface 1900 displayed on the display device 104 of the medical device 100. In this example, the user interface 1900 display a visual alarm 1902 which identifies the one or more actions performed in operation 1706 (e.g., reducing the brightness of the backlight light-emitting diodes (LEDSs) of the display device 104) to mitigate the temperature inside the medical device 100. The user interface 1900 further includes a plurality of tabs such as “Home”, “Patient”, “Review”, and “Settings”. Each tab when selected causes new information to be displayed on the display device 104. In the example provided in FIG. 19, the “Home” tab is shown as selected on the user interface 1900, causing a plurality of physiological variables such as non-invasive blood pressure (NIBP), blood oxygen saturation (SpO2), pulse rate, respiration rate (RR), body temperature, height, weight, and body mass index (BMI) to be displayed for a particular patient identified by patient name and patient ID.

Additionally, or alternatively, the alarm generated in operation 1708 can include a further visual alarm generated on an illumination unit 103, which is positioned on the top portion of the housing 102 of the medical device 100 (see also FIGS. 1-8). The illumination unit 103 allows for distant viewing of critical alarms such as when the internal temperature of the medical device 100 exceeds a threshold. The critical alarms generated on the illumination unit 103 can be viewed across a 360 degree field of view around the medical device 100. In such examples, both the visual alarm 1902 is displayed on the display device 104, and a further visual alarm is generated on the illumination unit 103 to indicate a critical status of the medical device 100 such as when the internal temperature inside the medical device 100 exceeds a threshold.

The illumination unit 103 can include one or more light-emitting diodes (LEDs) that emit light that is visible through a translucent cover. As an illustrative example, the illumination unit 103 can emit light having a color (e.g., yellow or red) based on a severity of the alarm. Additionally, or alternatively, the illumination unit 103 can emit light flashes based on a predetermined pattern associated with the severity of the alarm.

Additionally, or alternatively, the alarm generated in operation 1708 can include an audio alarm generated by a speaker 2130 (see FIG. 21) of the medical device 100. The audio alarm can notify the user that the temperature inside the medical device 100 exceeds a threshold. The audio alarm may also identify the one or more actions performed in operation 1706.

After the alarm is generated in operation 1708, the method 1700 can return to operation 1702 of monitoring the internal temperature of the medical device 100. Thereafter, the method 1700 can include repeating the operations 1704-1708.

When repeating the operations 1704-1708, the method 1700 can include determining in operation 1704 that the internal temperature of the medical device 100 no longer exceeds a predefined threshold that was previously exceeded. This can occur as a desired result from the action previously performed in operation 1706 to mitigate the heat inside the medical device 100. In such examples, the method 1700 can include reversing the action previously performed in operation 1706. For example, the method 1700 can include performing one or more actions such as increasing the clock speed of the processing device 2102 to reverse a previous decrease of the clock speed of the processing device 2102; increasing the clock speed of the GPU 2154 to reverse a previous decrease of the clock speed of the GPU 2154; increasing the charge current supplied by the charge circuit 126 for recharging the rechargeable battery 106 to reverse a previous decrease in the charge current; and/or increasing the brightness of the display device 104 to reverse a previous decrease in the brightness of the display device 104.

In some instances, when repeating the operations 1704-1708, the method 1700 can include determining in operation 1704 that the internal temperature exceeds a second predefined threshold that is higher than a first predefined threshold that was previously exceeded. In such examples, operation 1706 can include escalating the one or more actions to lower the internal temperature. For example, operation 1706 when repeated for a second time can include performing a further reduction in the clock speed of the processing device 2102; performing a further reduction in the clock speed of the GPU 2154; performing a further reduction in the charge current supplied by the charge circuit 126 for recharging the rechargeable battery 106; and/or performing a further reduction in the brightness of the display device 104. In some examples, operation 1706 can be repeated multiple times to provide multiple escalations of the one or more actions to lower the internal temperature of the medical device 100.

In some examples, when operation 1706 is repeated, the actions 1802-1808 are performed in a predetermined order. The predetermined order can range from actions that cause the least amount of interference with the operation of the medical device 100 to actions that cause the most amount of interference with the operation of the medical device 100. As an example, when operation 1706 is performed a first time, the action performed in operation 1706 includes reducing the clock speed of the processing device 2102; when operation 1706 is performed a second time, the action performed in operation 1706 includes reducing the clock speed of the GPU 2154; when operation 1706 is performed a third time, the action performed in operation 1706 includes reducing a charge current of the charge circuit 126 for recharging the rechargeable battery 106; and when operation 1706 is performed a fourth time, the action performed in operation 1706 includes reducing the brightness of the display device 104.

As further shown in FIG. 17, when the method 1700 determines in operation 1704 that the internal temperature exceeds a maximum allowed limit, the method 1700 proceeds to an operation 1710 of shutting off the medical device 100. Operation 1710 is performed as a last resort in order to protect the components of the medical device 100 from permanent damage.

In view of the foregoing, the method 1700 is performed to mitigate the internal heat generated by the medical device 100 without using a fan to cool off the internal components of the medical device. Advantageously, this can significantly reduce the noise generated by the medical device 100. Additionally, the method 1700 can reduce particles such as dirt and dust entering into the housing 102, which can plug the intake vents 114 and the exhaust vents 116.

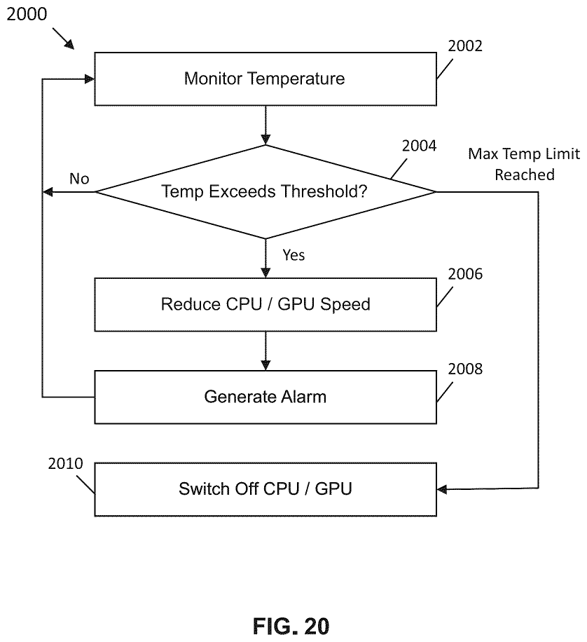

FIG. 20 schematically illustrates another example of a method 2000 of mitigating internal heat generated by the medical device 100. The method 2000 includes an operation 2002 of monitoring an internal temperature of the medical device 100. As disclosed above, the internal temperature can be monitored using an internal temperature sensor 2126 of the processing device 2102 (see FIG. 21), and/or the temperature sensor 2128 mounted on the main circuit board 120.

The method 2000 includes an operation 2004 of determining whether the internal temperature exceeds one or more thresholds. When it is determined that the internal temperature does not exceed the one or more thresholds (i.e., “No” in operation 2004), the method 2000 returns to operation 2002 of monitoring an internal temperature of the medical device 100.

Otherwise, when it is determined that the internal temperature does exceed the one or more thresholds (i.e., “Yes” in operation 2004), the method 2000 proceeds to an operation 2006 of reducing the clock speed of the processing device 2102 (i.e., first action 1802) and/or reducing the clock speed of the GPU 2154 of the display device 104 (i.e., second action 1804).

Next, the method 2000 can proceed to an operation 2008 of generating an alarm such as a visual or audio alarm to notify a user of the medical device 100 that the temperature inside the medical device 100 exceeds a threshold. Also, the visual or audio alarms generated in operation 2008 may also identify the action performed in operation 2006.

After completion of operation 2008, the method 2000 can repeat the operations 2002-2008 to continuously monitor the internal temperature of the medical device 100, and to continuously control the operation of the processing device 2102 and/or the GPU 2154 to mitigate excessive heat when generated inside the housing 102 of the medical device.

The method 2000 can include using predefined thresholds (e.g., passive trip points) to throttle back the clock speed of the processing device 2102 and/or the GPU 2154 by predetermined amounts based on the internal temperature of the medical device 100. Illustrative examples of the predefined thresholds and associated predetermined amounts of throttling back the clock speed of the processing device 2102 and/or the GPU 2154 are summarized in Table 1.

TABLE 1

| Temperature | CPU Speed | GPU Clock | GPU Speed | |
|---|---|---|---|---|
| (° C.) | (MHz) | Scaler | (MHz) | Alarm |
| <75 | 1200 | 64/64 | 800 | None |
| >75 | 1200 | 50/64 | 625 | None |
| >80 | 800 | 20/64 | 250 | Moderate |
| >86.3 | 800 | 5/64 | 62.5 | Critical |
| >95 | 0 | 0 | 0 | N/A |

The clock speed of the GPU 2154 is reduced by using a clock scaler. As an illustrative example, the GPU 2154 runs at a maximum speed of 800 MHZ. The clock scaler set by the processing device 2102 is N/64. Thus, a maximum clock speed of 800 MHz occurs when the clock scaler is set to 64/64, and a minimum clock speed of 12.5 MHz occurs when the clock scaler is set to 1/64. As summarized in Table 1, the processing device 2102 runs at a maximum speed of 1200 MHz and the GPU 2154 runs at a maximum speed of 800 MHZ (64/64) until reaching a first threshold of 75° C. When the internal temperature exceed the first threshold of 75° C., operation 2006 reduces the GPU speed to 625 MHz by setting the clock scaler to 50/64, while maintaining the maximum clock speed of the processing device 2102 at 1200 MHz.

As further shown in Table 1, when operation 2004 determines the internal temperature exceeds a second threshold of 80° C., operation 2006 includes reducing the clock speed of the processing device 2102 from 1200 MHz to 800 MHz. Additionally, when operation 2004 determines that the internal temperature exceeds the second threshold of 80° C., operation 2006 can include further reducing the clock speed of the GPU 2154 to 250 MHz by setting the clock scaler to 20/64. Additionally, when operation 2004 determines that the internal temperature exceeds the second threshold of 80° C., operation 2008 can include generating a moderate alarm.

As further shown in Table 1, when operation 2004 determines the internal temperature exceeds a third threshold of 86.3° C., operation 2006 includes maintaining the clock speed of the processing device 2102 at 800 MHZ, while further reducing the clock speed of the GPU to 62.5 MHz by setting the clock scaler to 5/64. Additionally, when operation 2004 determines that the internal temperature exceeds the third threshold of 86.3° C., operation 2008 can include generating a critical alarm having a higher priority than the moderate alarm (e.g., color red vs. color yellow, or blinking with a higher frequency).

When operation 2004 determines the internal temperature exceeds a maximum threshold of 95° C., operation 2006 includes reducing the clock speed of the processing device 2102 to zero, and reducing the clock speed of the GPU 2154 to zero, such that the medical device 100 is effectively turned off. This can prevent permanent damage to the internal components of the medical device 100 including the components mounted on the main circuit board 120.

No noticeable screen degradation is observed on the display device 104 until the clock scaler for the GPU 2154 is set to 3/64 of the maximum speed of 800 MHZ. Thus, as summarized in Table 1, the minimum value for the clock scaler is set to 5/64 before the GPU 2154 is turned off, which occurs when the maximum threshold of 95° C. is reached.

In view of the foregoing, the method 2000 is performed to mitigate the internal heat generated by the medical device 100 without using a fan to cool off the internal components of the medical device 100. Advantageously, this can significantly reduce the noise generated by the medical device. Additionally, the method 2000 can reduce particles such as dirt and dust entering into the housing 102, which can plug the intake vents 114 and the exhaust vents 116.

FIG. 21 illustrates an exemplary architecture of a computing device 2100 of the medical device 100. The computing device 2100 is used to execute the functionality of the medical device 100 described herein. The medical device 100 can include all or some of the elements described with reference to FIG. 21, with or without additional elements.

The computing device 2100 includes the processing device 2102. Examples of the processing device 2102 can include one or more central processing units (CPUs), digital signal processors, field-programmable gate arrays, and other types of electronic computing circuits. The processing device 2102 can be part of a processing circuitry having a memory for storing instructions which, when executed by the processing circuitry, cause the processing circuitry to perform the functionalities described herein.

As shown in FIG. 21, the processing device 2102 can include the internal temperature sensor 2126 that can be used to monitor the temperature of the processing device 2102. As discussed above, the temperature readings captured by the internal temperature sensor 2126 can be used by the computing device 2100 to determine whether to take action to mitigate the heat generated by the internal components of the medical device 100.

The computing device 2100 can also include the temperature sensor 2128, which can also be used to monitor the temperature inside the medical device 100. As shown in FIG. 11, the temperature sensor 2128 is mounted on the main circuit board 120. The temperature readings captured by the internal temperature sensor 2126 and/or the temperature sensor 2128 can be used by the computing device 2100 to determine whether to take action to mitigate the heat generated by the internal components of the medical device 100.

The computing device 2100 typically includes at least some form of computer-readable media. Computer-readable media includes any available media that can be accessed by the computing device 2100. By way of example, computer-readable media can include computer-readable storage media and computer-readable communication media.

Computer-readable storage media includes volatile and nonvolatile, removable, and non-removable media implemented in any device configured to store information such as computer-readable instructions, data structures, program devices, or other data. Computer-readable storage media includes, but is not limited to, random access memory (RAM), read only memory (ROM), electrically erasable programmable read only memory, flash memory or other memory technology, or any other medium that can be used to store the desired information and that can be accessed by the computing device 2100.

Computer-readable communication media embodies computer-readable instructions, data structures, program devices or other data in a modulated data signal such as a carrier wave or other transport mechanism and includes any information delivery media. Modulated data signal refers to a signal having one or more of its characteristics set or changed in such a manner as to encode information in the signal. By way of example, computer-readable communication media includes wired media such as a wired network or direct-wired connection, and wireless media such as acoustic, radio frequency, infrared, and other wireless media. Combinations of any of the above are also included within the scope of computer-readable media.

In the example of FIG. 21, the computing device 2100 includes a system memory 2104, and a system bus 2106 coupling various system components including the system memory 2104 to the processing device 2102. The system bus 2106 can include any type of bus structure including a memory bus, or memory controller, a peripheral bus, and a local bus.

The system memory 2104 can include a ROM 2108 and a RAM 2110. An input/output system containing routines to transfer information within the computing device 2100, such as during start up, can be stored in the ROM 2108. The system memory 2104 can include computer-readable media providing nonvolatile storage of computer-readable instructions (including application programs and data) for the computing device 2100.

The computing device 2100 can further include one or more secondary storage devices 2114 for storing digital data. The secondary storage devices 2114 are connected to the system bus 2106 by a secondary storage interface 2116. The secondary storage devices can include additional computer-readable media providing nonvolatile storage of computer-readable instructions (including application programs and data) for the computing device 2100.

A number of program devices can be stored in the system memory 2104 and/or the one or more secondary storage devices 2114. For example, an operating system 2118, one or more application programs 2120, and data 2122 can be stored in the system memory 2104 and/or the one or more secondary storage devices 2114. The system memory 2104 and the secondary storage devices 2114 are examples of computer-readable data storage devices.

The computing device 2100 includes input and output devices 2124. The input devices can include the display device 104, which as described above, is a touch sensitive touchscreen that receives inputs from a user of the medical device 100. Also, the input devices can include physical push buttons on the housing 102 of the medical device 100.

The input devices can further include the one or more peripheral sensors 2132 to measure the one or more physiological variables including blood pressure, blood oxygen saturation (SpO2), pulse rate, body temperature, and respiration rate. The input devices can further include the scanner to scan machine-readable codes attached to one or more objects.

The computing device 2100 can also include output devices such as the display device 104 and the speaker 2130. The display device 104 can be used by the computing device 2100 to display visual alarms such as when the temperature inside the housing 102 of the medical device 100 is determined to exceed a threshold. The speaker 2130 can be used by the computing device 2100 to generate audio alarms such as when the temperature inside the housing 102 of the medical device 100 is determined to exceed a threshold.

The input and output devices 2124 are connected to the processing device 2102 through an input/output interface 2138 coupled to the system bus 2106. The input and output devices 2124 can be connected by any number of input/output interfaces, such as a parallel port, serial port, game port, or a universal serial bus. Wireless communication between the input and output devices 2124 and the input/output interface 2138 is possible as well, and can include Wi-Fi, Bluetooth, infrared, 802.11a/b/g/n, or other wireless communications.

In some examples, the display device 104 is connected to the system bus 2106 via an interface, such as a video adapter 2142. The display device 104 includes touch sensors for receiving input from a user when the user touches the display device. Such sensors can be capacitive sensors, pressure sensors, or other touch sensors. The sensors detect contact with the display device 104, and also the location and movement of the contact over time. For example, a user can move a finger or stylus across the display device 104 to provide inputs.

The display device 104 further includes a graphics processing unit (GPU) 2154. The GPU 2154 provides graphics and video rendering on the display device 104, and can perform processing operations in parallel with the processing device 2102.

The computing device 2100 further includes a communication device 2146 configured to establish communication across a network 2152. In some examples, when used in a local area networking environment or a wide area networking environment (such as the Internet), the computing device 2100 is typically connected to the network 2152 through a network interface, such as a wireless network interface 2150. The wireless network interface 2150 can provide Wi-Fi connectivity for transfer and streaming of data such as measurements of physiological variables captured by the peripheral sensors 2132. In further examples, the wireless network interface 2150 provides Bluetooth connectivity. Other examples using other wired and/or wireless communications are possible. For example, the computing device 2100 can include an Ethernet network interface, or a modem for communicating across the network.

In further examples, the communication device 2146 provides short-range wireless communication. The short-range wireless communication can include one-way or two-way short-range to medium-range wireless communication. Short-range wireless communication can be established according to various technologies and protocols. Examples of short-range wireless communication include a radio frequency identification (RFID), a near field communication (NFC), a Bluetooth technology, a Wi-Fi technology, or similar wireless technologies.

The computing device 2100 is an example of programmable electronics, which may include one or more computing devices, and when multiple computing devices are included, such computing devices can be coupled together with a suitable data communication network so as to collectively perform the various functions, methods, or operations disclosed herein.

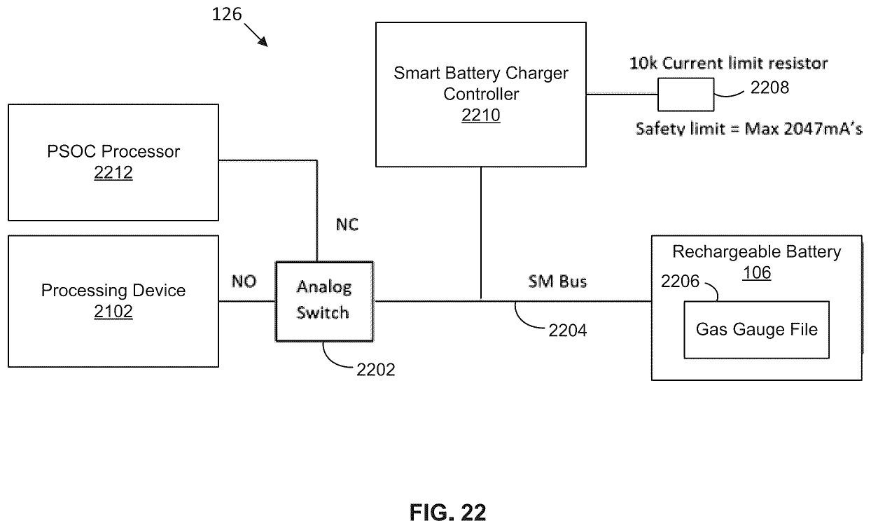

FIG. 22 schematically illustrates an example of the charge circuit 126 for the rechargeable battery 106 of the medical device 100. As described above, the charge circuit 126 can be mounted on the main circuit board 120. In some examples, the rechargeable battery 106 has a capacity of about 7 Ampere-hours (Ah). A maximum charge current of the rechargeable battery 106 is set by a battery gas gauge file 2206 and a fixed resistor 2208 on the main circuit board 120 that is connected to a smart battery charger controller 2210. In some examples, the maximum charge current of the rechargeable battery 106 is set at about 2 Amps. In some examples, the smart battery charger controller 2210 is a LTC4100 chip. The processing device 2102 can adjust the charge current of the rechargeable battery 106 of the medical device 100 from about 2 Amps to about 0 Amps (e.g., 0x07FF through 0x0000).

As shown in FIG. 22, the charge circuit 126 includes a switch 2202 on a system management (SM) bus 2204 that switches from the processing device 2102 to a programmable system on a chip (PSoC) 2212 depending on a status of the medical device 100. For example, the PSoC 2212 can be used when the medical device 100 is powered off and the processing device 2102 can be used when the medical device 100 is powered on. Either of the processing device 2102 or the PSoC 2212 can adjust the charge rate of the rechargeable battery 106.

The various embodiments described above are provided by way of illustration only and should not be construed to be limiting in any way. Various modifications can be made to the embodiments described above without departing from the true spirit and scope of the disclosure.

What is claimed is:

1. A medical device for capturing one or more physiological variables of a patient, the medical device comprising:
a housing;
a display device mounted on the housing;
ports on the housing for connecting one or more peripheral sensors;
at least one processing device; and
at least one computer readable data storage device storing software instructions that, when executed by the at least one processing device, cause the at least one processing device to:
receive data from the peripheral sensors;
process the data to calculate at least one physiological variable;
display the at least one physiological variable on the display device;
monitor an internal temperature inside the housing;
determine whether the internal temperature exceeds a predefined threshold; and
when the internal temperature exceeds the predefined threshold, perform one or more actions to lower the internal temperature.

2. The medical device of claim 1, wherein the one or more actions include at least one of reducing a clock speed of the at least one processing device and reducing a clock speed of a graphics processing unit of the display device.

3. The medical device of claim 2, wherein the clock speed of the at least one processing device is reduced from 1200 MHz to 800 MHz.

4. The medical device of claim 2, wherein the clock speed of the graphics processing unit is reduced from 800 MHz to 625 MHz, or from 625 MHz to 250 MHz, or from 250 MHz to 62.5 MHz.

5. The medical device of claim 2, wherein the instructions, when executed by the at least one processing device, further cause the at least one processing device to:
determine the internal temperature exceeds a maximum threshold; and
perform at least one of reducing the clock speed of the at least one processing device to zero and reducing the clock speed of the graphics processing unit to zero.

6. The medical device of claim 1, further comprising:
a rechargeable battery; and
wherein the one or more actions include reducing a charge current for recharging the rechargeable battery.

7. The medical device of claim 1, wherein the one or more actions include reducing a brightness of the display device.

8. The medical device of claim 1, wherein the at least one processing device includes an internal temperature sensor, and wherein the internal temperature sensor of the at least one processing device is used to monitor the internal temperature.

9. The medical device of claim 1, further comprising:
a temperature sensor for monitoring the internal temperature inside the housing.

10. The medical device of claim 1, further comprising:
a main circuit board mounted inside the housing;
intake vents positioned across a bottom surface of the housing; and
exhaust vents positioned toward a top portion of the housing, wherein the intake and exhaust vents cause a thermal flow of air that passes around the main circuit board causing convection of heat away from the main circuit board toward the exhaust vents.

11. The medical device of claim 10, further comprising:
a handle on the housing, wherein the exhaust vents are set back and positioned under the handle such that the handle provides a canopy over the exhaust vents.

12. The medical device of claim 10, further comprising:
a plurality of components arranged on the main circuit board, wherein the plurality of components are arranged in a layout having components generating a largest amount of heat positioned toward a top portion of the main circuit board, and components generating a minimal amount of heat or no heat positioned toward a bottom portion of the main circuit board.

13. The medical device of claim 12, wherein the plurality of components include a heat sink mounted on the main circuit board, the heat sink configured to transfer thermal energy from the main circuit board to the thermal flow of air surrounding the main circuit board.

14. The medical device of claim 1, further comprising:
a thermometer module integrated with the medical device, the thermometer module including a handheld probe for taking a temperature reading of the patient.

15. The medical device of claim 1, further comprising:
an illumination unit positioned on a top portion of the housing; and
wherein the instructions, when executed by the at least one processing device, further cause the at least one processing device to:
generate an alarm on the illumination unit to indicate the internal temperature exceeds the predefined threshold, the illumination unit allowing the alarm to be viewed across a 360 degree field of view around the medical device.

16. The medical device of claim 1, wherein the housing does not include a fan for lowering the internal temperature inside the housing.

17. A method of capturing one or more physiological variables of a patient, the method comprising:
receiving data from a peripheral sensor;
processing the data to calculate a physiological variable by using a computing device mounted inside a housing;
displaying the physiological variable on a display device;
monitoring an internal temperature inside the housing;
determining whether the internal temperature exceeds a predefined threshold;
performing one or more actions to lower the internal temperature when the internal temperature exceeds the predefined threshold; and
generating an alarm identifying the one or more actions performed to lower the internal temperature.

18. The method of claim 17, wherein the one or more actions include at least one of reducing a charge current for recharging a rechargeable battery, reducing a brightness of the display device, reducing a clock speed of the at least one processing device, and reducing a clock speed of a graphics processing unit of the display device.

19. The method of claim 18, wherein the clock speed of the at least one processing device is reduced from 1200 MHz to 800 MHZ, and the clock speed of the graphics processing unit is reduced from 800 MHz to 625 MHz or from 625 MHz to 250 MHz or from 250 MHz to 62.5 MHz.

20. The method of claim 18, further comprising:
determining the internal temperature exceeds a maximum threshold; and
performing at least one of reducing the clock speed of the at least one processing device to zero and reducing the clock speed of the graphics processing unit to zero.
